(19) United States
(12) Patent Application Publication
(10) Pub. No.: US 2023/0364133 A1
(43) Pub. Date: Nov. 16, 2023

(54) DIETARY SUPPLEMENT COMPOSITIONS

(71) Applicant: Melaleuca, Inc., Idaho Falls, ID (US)

(72) Inventors: Nasser A. Fredj, Ammon, ID (US); Clinton Dahlberg, Idaho Falls, ID (US); Hassen Nasri, Ammon, ID (US); Jordan Joy, Idaho Falls, ID (US); Luping Yan, Ammon, ID (US)

(21) Appl. No.: 18/195,716

(22) Filed: May 10, 2023

Related U.S. Application Data
(60) Provisional application No. 63/340,269, filed on May 10, 2022.

(51) Int. Cl. A61K 33/30; A61K 31/716; A61K 31/352; A61K 31/07; A61K 31/375; A61K 31/593; A61K 36/489; A61K 36/05

(52) U.S. Cl. CPC A61K 33/30; A61K 31/07; A61K 31/352; A61K 31/375; A61K 31/593; A61K 31/716; A61K 36/05; A61K 36/489

(57) ABSTRACT

This document provides dietary supplement compositions. For example, dietary supplement compositions having beta glucan, quercetin, and one or more of vitamin A, vitamin C, vitamin D, and zinc are provided herein.

### CROSS-REFERENCE TO RELATED APPLICATIONS

This application claims priority from U.S. Provisional Application Serial No. 63/340,269, filed May 10, 2022. The disclosure of the prior application is considered part of (and is incorporated by reference in) the disclosure of this application.

### BACKGROUND

### 1. Technical Field

This document relates to the field of dietary supplements. For example, this document relates to dietary supplement compositions useful for increasing immunity in humans or other mammals.

### 2. Background Information

Many people desire improved health and well-being, particularly with their immune health and function. According to the CDC in 2020, two of the 10 leading causes of death in the United States were directly related to immune health and function (https://www.cdc.gov/nchs/fastats/deaths.htm). COVID-19, and influenza or pneumonia, were the third and ninth most deadly conditions, respectively. With the exception of unintentional injuries, all of the remaining leading causes of death are indirectly due to poor immune health. This fact was on global display with the COVID-19 pandemic, with over 90% of deaths associated with at least one other pre-existing comorbidity and over 1000-fold greater likelihood of death with at least one comorbidity versus the absence of comorbidities. Accordingly, there is a need for a stronger immune system throughout the world’s human population.

### SUMMARY

This document provides dietary supplement compositions. For example, this document provides dietary supplement compositions useful for human or animal consumption. The dietary supplement compositions provided herein can improve immunity. In general, the dietary supplement compositions provided herein include beta glucan, quercetin, and one or more of vitamin A, vitamin C, vitamin D, and zinc.

In general, the dietary supplement compositions described herein can increase immune function (e.g., production and/or action of cells that fight disease or infection) of a subject that was administered the composition. In some cases, the compositions provided herein can include quercetin having increased bioavailability (e.g., bioavailability greater than 1, such as bioavailability from about 2 to about 100). In some cases, the beta-glucan and quercetin in the compositions described herein can have a synergistic effect on the immune system.

In one aspect, this document features dietary supplement compositions that can include, consist of, or consist essentially of beta glucan, quercetin, and one or more of vitamin A, vitamin C, vitamin D, and zinc. The composition can include the beta glucan in the form of β-(1,3)-glucan. The composition can include the quercetin complexed to one or more phospholipids. The composition can include the quercetin in a blend comprising a Sophora japonica extract and one or more phosopholipids. The composition can include vitamin A. The composition can include vitamin C. The composition can include vitamin D. The composition can include zinc. The composition can include vitamin A, vitamin C, vitamin D, and zinc. The composition can include zinc in the form of an amino acid chelated zinc.

In another aspect, this document features dietary supplement compositions that can include, consist of, or consist essentially of beta glucan, quercetin, vitamin D, vitamin A, vitamin C, and zinc. The composition can include the beta glucan in the form of β-(1,3)-glucan. The composition can include the quercetin complexed to one or more phospholipids. The composition can include the quercetin in a blend comprising a Sophora japonica extract and one or more phosopholipids. The composition can include zinc as a zinc-amino acid compound. The composition can include zinc as a zinc-amino acid compound conjugated with a polysaccharide. The composition can include the beta glucan in an amount of about 35 mg to about 1500 mg. The composition can include the quercetin in an amount of about 5 mg to about 1500 mg. The composition can include the vitamin A in an amount of about 25 mcg RAE to about 1500 mcg RAE. The composition can include the vitamin C in an amount of about 9 mg to about 2000 mg. The composition can include the vitamin D in an amount of about 0.005 mg to about 0.1 mg. The composition can include the zinc in an amount of about 1 mg to about 75 mg. The composition can include the quercetin has a bioavailability of greater than 1, or about 2 to about 100, or about 25 to about 75, or about 40 to about 60. The composition can include beta glucan, wherein the beta glucan was isolated from algae. In some cases the algae is Euglena gracillis algae. The composition can be in the form of a tablet or capsule.

Also provided herein are dietary supplement compositions that can include, consist of, or consist essentially of beta glucan in an amount of about 35 mg to about 1500 mg, quercetin in an amount of about 5 mg to about 1500 mg, vitamin A in an amount of about 25 mcg RAE to about 1500 mcg RAE mg, vitamin C in an amount of about 9 mg to about 2000 mg, vitamin D in an amount of about 0.005 mg to about 0.1 mg, and zinc in an amount of about 1 mg to about 75 mg. The composition can include the beta glucan in an amount of about 100 mg to about 300 mg. The composition can include the quercetin in an amount of about 5 mg to about 200 mg. The composition can include the vitamin A in an amount of about 400 mcg RAE to about 500 mcg RAE. The composition can include the vitamin C in an amount of about 10 mg to about 100 mg. The composition can include the vitamin D in an amount of about 0.009 mg to about 0.05 mg. The composition can include the zinc in an amount of about 5 mg to about 50 mg. The composition can include the zinc in the form of an amino acid chelated zinc. The composition can include the quercetin complexed to one or more phospholipids. The composition can include the quercetin in a blend comprising a Sophora japonica extract and one or more phosopholipids. The composition can include the beta glucan in the form of β (1,3)-glucan.

In still another aspect, this document features dietary supplement compositions that can include at least about 35 mg of beta glucan, at least about 5 mg of quercetin, at least about 25 mcg RAE of vitamin A, at least about 9 mg of vitamin C, at least about 0.005 mg of vitamin D, and at least about 1 mg of zinc.

As used herein, the term “about” when used to refer to weight % in a composition means ± 10% of the reported weight %. As used herein, the term “about” when used to refer to measured characteristics of the composition means ± 20% of the reported value.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DETAILED DESCRIPTION

This document provides dietary supplement compositions including beta glucan, quercetin, and one or more of vitamin A, vitamin C, vitamin D, and zinc. For example, a dietary supplement composition provided herein can include beta glucan, quercetin, vitamin A, vitamin C, vitamin D, and zinc. In some cases, a dietary supplement composition provided herein can contain beta glucan, quercetin, and vitamin A; beta glucan, quercetin, and vitamin C; beta glucan, quercetin, and vitamin D; beta glucan, quercetin, and zinc; beta glucan, quercetin, vitamin A, and vitamin C; beta glucan, quercetin, vitamin A, and vitamin D; beta glucan, quercetin, vitamin A, and vitamin D; beta glucan, quercetin, vitamin A, and zinc; beta glucan, quercetin, vitamin C, and vitamin D; beta glucan, quercetin, vitamin C, and zinc; beta glucan, quercetin, vitamin D, and zinc; beta glucan, quercetin, vitamin A, vitamin C, and vitamin D; beta glucan, quercetin, vitamin A, vitamin C, and zinc; beta glucan, quercetin, vitamin A, vitamin D, and zinc; or beta glucan, quercetin, vitamin C, vitamin D, and zinc.

The dietary supplement compositions provided herein contain quercetin, a flavonoid that has antioxidant properties, anti-inflammatory properties, and immune-enhancing functions. For example, quercetin is capable of stimulating T-helper 1, T-helper 2, and T-regulatory cell cytokine production, which may enhance both the defensive action of T-killer cells as well as the feedback signal to “turn off” cytokine production once the pathogen is no longer present (e.g., to prevent cytokine storm). Quercetin also has antiviral activity that is mediated by inhibiting viral proteases.

Quercetin can have poor absorption when administered orally or by topical application, partly due to bacterial degradation of the phenol moiety of the molecule, and also due to complex formation with other substances present in the gastrointestinal tract. When quercetin is complexed to a phospholipid to increase its overall lipophilic character, however, it can be much more readily absorbed after oral administration, thereby increasing the therapeutic effectiveness of the quercetin. Thus, in some embodiments, the dietary supplement compositions provided herein can include quercetin complexed to one or more phospholipids. The phospholipids that can be complexed to quercetin can either be natural or synthetic in nature. In some embodiments, the phospholipid molecules can include acyl residues that are the same or different from one another. Non-limiting examples of acyl residues include palmitic acid, stearic acid, oleic acid, linoleic acid, and linolenic acid. In some embodiments, quercetin can be complexed with a glycerophospholipid. Non-limiting examples of glycerophospholipids for complexing quercetin include phosphatidylcholine, phosphatidylethanolamine, phosphatidylserine, phosphatidic acid, phosphatidylinositol, phosphatidylinositol phosphate, bisphosphate, trisphosphate, a phosphingolipid (e.g., ceramide phosphorylcholine, ceramide phosphorylethanolamine, or ceramide phosphorylipid), and phosphatidylglycerol cardiolipin.

Any appropriate ratio of quercetin to phospholipid can be used. In some embodiments, the molar ratio of quercetin to the one or more phospholipids can be about 1:0.8 to about 1:5. For example, the molar ratio of quercetin to the one or more phospholipids can be about 1:0.8 to about 1:0.9, about 1:0.9 to about 1:1, about 1:1 to about 1:2, about 1:2 to about 1:3, about 1:3 to about 1:4, about 1:5 to about 1:5, or about 1:1 to about 1:1.5. Any appropriate source of quercetin can be used in the compositions provided herein. In some embodiments, for example, the composition can include the quercetin in a blend comprising a Sophora japonica extract (e.g., wherein the Sophora japonica extract includes quercetin) and one or more phosopholipids. In some embodiments, the quercetin blend can include purified quercetin and one or more phospholipids.

In some embodiments, the quercetin in the compositions provided herein has a bioavailability of at least 1. For example, the quercetin can have a bioavailability of about 2 to about 100, or about 5 to about 95, or about 10 to about 90, or about 20 to about 80, or about 30 to about 70, or about 40 to about 60, or about 50.

The compositions provided herein can contain any appropriate amount of quercetin. In some embodiments, for example, a composition can include quercetin in an amount of at least about 5 mg. In some embodiments, a composition can include quercetin in an amount of about 5 mg to about 1500 mg. For example, quercetin can be present in the composition in an amount of about 5 mg to about 1250 mg, about 5 mg, to about 1000 mg, about 5 mg to about 750 mg, about 25 mg to about 500 mg, about 5 mg to about 500 mg, about 5 mg to about 200 mg, about 10 mg to about 350 mg, about 15 mg to about 300 mg, about 20 mg to about 200 mg, about 25 mg to about 150 mg, about 25 mg to about 100 mg, or about 25 mg to 75 mg. In some embodiments, quercetin can be present in the composition in an amount of about 50 mg.

The dietary supplement compositions provided herein also include beta glucan. The beta glucan included in the compositions provided herein can include one or more forms of beta glucan, such as β-((1,3)(1,4))-glucan, β-((1,3)(1,6))-glucan, β-(1,4)-glucan, and β-(1,3)-glucan. In some embodiments, the beta glucan is in the form of (3-(1,3)-glucan. β-(1,3)-glucan and β-(1,3)(1,6)-glucan in particular can provide immune-enhancing properties, but only (3-(1,3)-glucan can provide these immune-enhancing properties at low doses (e.g., 200 mg or less).

The compositions provided herein can contain any appropriate amount of beta glucan. In some embodiments, for example, the compositions can include beta glucan in an amount of at least about 35 mg. In some embodiments, the compositions can include beta glucan in an amount of about 35 mg to about 1500 mg. For example, beta glucan can be present in a composition in an amount of about 50 mg to about 1250 mg, about 50 mg to about 1000 mg, about 100 mg to about 750 mg, about 100 mg to about 500 mg, or about 150 mg to about 350 mg, or about 150 mg to about 300 mg, or about 150 mg to about 250 mg, or about 150 mg to about 220 mg, or about 175 mg to about 200 mg. In some embodiments, beta glucan can be present in the composition in an amount of about 190 mg.

In some embodiments, the compositions provided herein include vitamin A. The vitamin A in the dietary supplement compositions provided herein can include, without limitation, one or more of β-carotene, retinol, retinaldehyde, retinoic acid, and salts thereof. For example, the vitamin A can include one or more of β-carotene, retinyl palmitate, or retinyl acetate. In some embodiments, the composition includes vitamin A as retinyl palmitate. In some embodiments, the composition includes vitamin A as β-carotene.

When vitamin A is present in the compositions herein, the compositions can include vitamin A in an amount of at least about 5% of the U.S. Recommended Daily Intake (USRDI) of vitamin A. The U.S. Recommended Daily Intake (USRDI) for vitamins and minerals are defined and set forth in the Recommended Daily Dietary Allowance-Food and Nutrition Board, National Academy of Sciences-National Research Council. In some embodiments, vitamin A is present in the compositions provided herein in an amount of about 10% to about 200% of the USRDI of vitamin A. For example, vitamin A can be present in the compositions provided herein in an amount of about 5% to about 150%, about 10% to about 150%, or about 20% to 125%, or about 30% to about 100%, or about 30% to about 75% or about 50% of the USRDI of vitamin A.

In some embodiments, when vitamin A is present in a composition provided herein, the composition can include vitamin A in an amount of at least about 25 mcg RAE, wherein the term “mcg RAE” refers to micrograms of retinol activity equivalents. For example, vitamin A can be present in the compositions provided herein in an amount of about 25 mcg RAE to about 1500 mcg RAE, about 50 mcg RAE to about 1000 mcg RAE, about 100 mcg RAE to about 1000 mcg RAE, about 200 mcg RAE to about 750 mcg RAE, about 300 mcg RAE to about 750 mcg RAE, about 400 mcg RAE to about 500 mcg RAE. In some cases, vitamin A is present in the composition in an amount of about 400 mcg RAE to about 500 mcg RAE. In some embodiments, vitamin A is present in the composition in an amount of about 450 mcg RAE.

In some embodiments, the compositions provided herein include vitamin C. The vitamin C included in the dietary supplement compositions provided herein can include ascorbic acid or salts thereof. For example, the vitamin C can include, without limitation, one or more of ascorbic acid, sodium ascorbate, calcium ascorbate, potassium ascorbate, magnesium ascorbate, ascorbyl palmitate, and ascorbyl stearate. In some embodiments, the composition includes vitamin C as ascorbic acid. In some embodiments, the composition includes vitamin C as sodium ascorbate. In some embodiments, the composition includes one or more natural sources of vitamin C (e.g., lemon, orange, tomato, or acerola).

When vitamin C is present in the compositions provided herein, the compositions can include vitamin C in an amount of at least about 5% of the USRDI of vitamin C. In some embodiments, vitamin C can be present in the compositions provided herein in an amount of about 10% to about 500% of the USRDI of vitamin C. For example, vitamin C can be present in the composition in an amount of about 10% to about 350%, about 10% to about 250%, about 10% to about 150%, about 15% to about 125%, or about 20% to about 100%, about 25% to about 75%, or about 30% of the USRDI of vitamin C.

In some embodiments when vitamin C is present in a composition provided herein, the composition can include vitamin C in an amount of at least about 9 mg. For example, a composition provided herein can include vitamin C in an amount of about 9 mg to about 2000 mg. In some cases, vitamin C is present in the composition in an amount of about 10 mg to about 100 mg. In some embodiments, the composition includes vitamin C in an amount of about 27 mg.

In some embodiments, the compositions provided herein include vitamin D. The vitamin D in the dietary supplement compositions herein can include, without limitation, cholecalciferol or ergocalciferol. For example, the vitamin D can include one or both of cholecalciferol and ergocalciferol. In some embodiments, the composition includes vitamin D as cholecalciferol. In some embodiments, the composition includes vitamin D as cholecalciferol and ergocalciferol.

When vitamin D is present in the compositions provided herein, the compositions can include vitamin D in an amount of at least about 5% of the USRDI of vitamin D. For example, vitamin D can be present in the compositions provided herein in an amount of about 5% to about 200% of the USRDI of vitamin D (e.g., about 5% to about 150%, about 10% to about 100%, about 25% to about 150%, or about 15% to about 125%, about 50% to about 150%, about 75% to about 125%, or about 100% of the USRDI of vitamin D.

In some embodiments when vitamin D is present in the compositions provided herein, the compositions can include vitamin D in an amount of at least about 0.001 mg. For example, the compositions provided herein can include vitamin D in an amount of about 0.001 mg to about 0.1 mg (e.g., about 0.005 mg to about 0.1 mg, about 0.009 mg to about 0.05 mg, or about 0.02 mg).

In some embodiments, the compositions provided herein can include zinc. In some cases, the zinc within the compositions provided herein can be in the form of a zinc-amino acid compound in which zinc is chelated with (e.g., forms a salt with) an amino acid. In some embodiments, the zinc-amino acid compound can be an amino acid chelated zinc. In some embodiments, the zinc-amino acid compound can be a zinc-amino acid salt. Any suitable amino acid can be used to form a chelate with the zinc to create a zinc-amino acid compound. In some embodiments, the amino acid portion of a zinc-amino acid compound can be one or more natural or unnatural amino acids. For example, an amino acid can be a natural amino acid. As used herein, the term “natural” amino acid refers to one of the twenty commonly occurring amino acids. Natural amino acids can be in their D or L form. For example, a natural amino acid can be selected from the group consisting of L-alanine, L-arginine, L-asparagine, L-aspartic acid, L-cysteine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, L-valine, and mixtures thereof. In some cases, an amino acid is selected from L-glycine and L-aspartic acid.

In some cases, the zinc included in the compositions provided herein can be in the form of a zinc-amino acid compound complexed with a polysaccharide. Any appropriate polysaccharide can be conjugated with the zinc-amino acid compound to form a complex. For example, the polysaccharide can include a cellulose derivative, polyhexose, polypentose, polydextrose, starch, polygalactan, polymannan, chitin, chitosan, chondroitin, polyfructose, polyfructose (e.g., inulin), pectin, and derivatives thereof. In some embodiments, inulin having a degree of polymerization ranging from about 2 to about 100 (e.g., about 2-10; about 12-15; about 20-30; about 25-45; about 30-40; about 50-75; about 45-65; about 50-55; about 70-80; about 75-90; or about 92-100) can be conjugated with a zinc-amino acid compound to form a complex.

A complex of a zinc-amino acid compound and a polysaccharide can be prepared using any appropriate method. For example, a zinc-amino acid polysaccharide complex can be prepared by heating a composition including water, one or more zinc-amino acid compounds, and one or more polysaccharides at a temperature from about 100° F. to about 180° F. (e.g., about 110° F. to about 160° F., or about 120° F. to about 150° F.; or about 100° F.; about 110° F.; about 120° F.; about 125° F.; about 130° F.; about 140° F.; about 145° F.; about 150° F.; about 160° F.; about 165° F.; about 170° F.; about 175° F.; and about 180° F.). In some embodiments, the composition can be heated at from about 140° F. to about 180° F. In some embodiments, the composition can be heated at about 160° F. In some embodiments, the composition can be heated for from about 5 minutes to about 30 minutes (e.g., about 10 minutes to about 25 minutes, about 15 minutes to 20 minutes, or about 5 minutes; about 10 minutes; about 15 minutes; about 20 minutes; about 25 minutes; and about 30 minutes). In some embodiments, the composition can be heated for about 20 minutes. In some embodiments, the complex can be dried, for example, to a moisture content of less than about 15% (e.g., less than about 14%, less than about 12%, less than about 10%, less than about 8%, less than about 5%, and less than about 2%) following heating.

A complex of the zinc-amino acid compound and polysaccharide can be prepared using a ratio of zinc-amino acid compound to polysaccharide ranging from, for example, 10:1 to 1:10 (e.g., 10:1; 8:1, 7:1, 6:1; 5:1; 4:1; 3:1; 2:1; 1:1.5; 1:1; 1:1.5; 1:2; 1:3; 1:4; 1:5; 1:6; and 1:10). For example, a ratio of zinc-amino acid compound to polysaccharide can be 5:1 or 1:1. The zinc-amino acid/polysaccharide complex can contain any appropriate form of zinc, any appropriate amino acid, and any appropriate polysaccharide. For example, the zinc-amino acid compound/polysaccharide complex can include one or more of zinc sulfate, zinc picolinate, zinc citrate, zinc gluconate, zinc acetate, zinc glycerate, and zinc monomethionine. The amino acid in a zinc-amino acid /polysaccharide complex can include, without limitation, one or more of methionine, glycine, aspartic acid, alanine, arginine, asparagine, cysteine, glutamic acid, glutamine, histidine, isoleucine, leucine, lysine, phenylalanine, proline, serine, threonine, tryptophan, tyrosine, and valine. The polysaccharide in a zinc-amino acid/polysaccharide complex can be selected from, without limitation, cellulose, polyhexoses, polypentoses, polydextrose, starch, polygalactan, polymannan, chitin, chitosan, chondroitin, polyfructose, inulin, and pectin. In some cases, a zinc-amino acid compound/polysaccharide complex can include zinc sulfate, glycine, and inulin, optionally in combination with sodium citrate. The zinc-amino acid compound/polysaccharide complex can include, for example, about 30 wt% to about 60 wt% zinc sulfate, about 25 wt% to about 50 wt% glycine, about 7 wt% to about 13 wt% inulin, and about 7 wt% to about 11 wt% sodium citrate (e.g., about 35 wt% to about 53 wt% zinc sulfate, about 29 wt% to about 45 wt% glycine, about 8.5 wt% to about 11.5 wt% inulin, and about 8 wt% to about 10 wt% sodium citrate, or about 35.3 wt% to about 53% zinc sulfate, about 29.6 wt% to about 44.3 wt% glycine, about 8.9 wt% to about 10.9 wt% inulin, and about 8.1 wt%, to about 9.9 wt% sodium citrate). In some cases, a zinc-amino acid/polysaccharide complex can further include sodium alginate (e.g., where the sodium alginate is a coating over the zinc-amino acid/polysaccharide complex). Any other suitable coating material also can be included (e.g., stearic acid, carbomer copolymer, shellac, hypromellose, carboxymethylcellulose sodium, carrageenan, cellaburate, ethylcellulose, glyceryl monooleate, pregelatinized modified starch, glyceryl monostearate, guar gum, hydroxypropyl betadex, hydroxypropyl cellulose, polyethylene oxide, polyvinyl acetate dispersion, pregelatinized starch, xanthan gum, alginic acid, locust bean gum, hydrogenated vegetable oil, monoglycerides, and/or diglycerides), in any appropriate amount. For example, a zinc-amino acid compound/polysaccharide complex can further include about 2 wt% to about 15 wt% (e.g., about 2 wt% to about 5 wt%, about 5 wt%, to about 8 wt%, about 8 wt%, to about 10 wt%, about 10 wt% to about 12 wt%, or about 12 wt% to about 15 wt%) sodium alginate.

In some embodiments, the dietary supplement compositions can include two or more different zinc-amino acid compounds or zinc-amino acid/polysaccharide complexes. For example, in some cases, the zinc-amino acid compound or zinc-amino acid/polysaccharide complex can include a zinc-amino acid compound that contains aspartate and glycinate (e.g., about 75% aspartate and about 25% glycinate). In some cases, the zinc-amino acid compound/polysaccharide complex can include polyfructose. In some cases, the zinc-amino acid compound/polysaccharide complex can include inulin having a degree of polymerization ranging from about 2 to about 100. In some cases, the zinc-amino acid compound/polysaccharide complex can include inulin having a degree of polymerization of about 12-15.

The dietary supplement compositions provided herein can be formulated in any appropriate manner. For example, the compositions provided herein can be in the form of a liquid, solution, suspension, tablet, powder, cream, mist, atomized vapor, aerosol, soft gelatin capsule, hard gelatin capsule, gums, lozenges, candy, sachet, gel, confectionary, shake, bar, or supplemented food.

In some cases, the dietary supplement compositions provided herein can be formulated for oral administration and can include one or more suitable excipients, flavorings, colorants, and other ingredients. For oral administration, tablets or capsules can be prepared with pharmaceutically acceptable excipients such as binding agents, fillers, lubricants, disintegrants, or wetting agents. In some cases, tablets can include a coating (e.g., a polymer or polysaccharide-based coating with or without plasticizers and/or pigments). Liquid preparations for oral administration can take the form of, for example, solutions, syrups, or suspension, or they can be presented as a dry product for constitution with saline or other suitable liquid vehicle before use. In some cases, liquid preparations can contain pharmaceutically acceptable additives such as suspending agents, emulsifying agents, non-aqueous vehicles, preservatives, buffer salts, flavoring agents, coloring agents, and sweetening agents as appropriate. Preparations for oral administration can be suitably formulated to give controlled release of one or more compounds. In some cases, tablets or capsules can be coated with a methacrylic acid copolymer (e.g., EUDRAGIT^{®} L100-55 or EUDRAGIT^{®} S100) for release beyond the stomach (e.g., in the intestine, colon, or both).

In some cases, dietary supplement compositions provided herein can contain a pharmaceutically acceptable carrier for administration to a mammal, including, without limitation, sterile aqueous or non-aqueous solutions, suspensions, and emulsions. Examples of non-aqueous solvents include, without limitation, propylene glycol, polyethylene glycol, vegetable oils, and organic esters. Aqueous carriers include, without limitation, water, alcohol, saline, and buffered solutions. Pharmaceutically acceptable carriers also can include physiologically acceptable aqueous vehicles (e.g., physiological saline) or other carriers appropriate for oral administration.

In some cases, the dietary supplement compositions provided herein can be in the form of a capsule or tablet, by way of an example only, configured to have a unit dosage equal to the daily desired dosage for a particular mammal. For example, if a mammal is to be administered a dose of 100 mg of a particular agent, each tablet can include about 100 mg in weight of that agent. The compositions provided herein can be formulated for administration to any appropriate mammal. For example, the compositions provided herein can be for administration to humans or other mammals (e.g., dogs, cats, mice, rats, rabbits, cows, horses, pigs, or sheep). In some cases, a total daily dose may be prepared and administered in the form of one or more dosage units (e.g., two tablets or capsules, three tablets or capsules, four tablets or capsules, five tablets or capsules, or six tablets or capsules). For instance, in some cases, an exemplary dietary supplement composition can be provided in three separate tablets or capsules.

Also provided herein are methods for providing a subject (e.g., a mammal such as a human, mouse, rat, rabbit, dog, cat, cow, horse, pig, or sheep) with beta glucan, quercetin, and one or more of vitamin A, vitamin C, vitamin D, and zinc. In some cases, the methods can include administering to a subject a composition including beta glucan, quercetin, and one or more of vitamin A, vitamin C, vitamin D, and zinc, where the subject exhibits increased immunity as compared to a corresponding subject not administered the composition as described herein. For example, the composition can include beta glucan in the form of β-(1,3)-glucan to increase immunity of the subject that has been administered the composition. In some cases, the composition can include quercetin having a bioavailability of greater than 1 (e.g., about 25 to about 75) to increase immunity of the subject that has been administered the composition.

In some cases, the methods provided herein can be used to treat a disease or clinical condition (e.g., an immune related disorder such as, without limitation, Addison disease, Celiac disease, Dermatomyositis, Graves’ disease, Hashimoto thyroiditis, multiple sclerosis, myasthenia gravis, pernicious anemia, reactive arthritis, rheumatoid arthritis, Sjogren syndrome, type 1 diabetes, psoriasis, irritable bowel syndrome, asthma, ataxia telangiectasia, autoimmune polyglandular syndrome, Burkitt lymphoma, DiGeorge syndrome, chronic myeloid leukemia, or human immunodeficiency virus (HIV)/acquired immune deficiency syndrome (AIDS)) in a subject, by increasing the immunity of the subject.

In some cases, the methods provided herein can be used to reduce the likelihood of developing a disease or clinical condition (e.g., an immune related disorder such as, without limitation, Addison disease, Celiac disease, Dermatomyositis, Graves’ disease, Hashimoto thyroiditis, multiple sclerosis, myasthenia gravis, pernicious anemia, reactive arthritis, rheumatoid arthritis, Sjogren syndrome, type 1 diabetes, or psoriasis, irritable bowel syndrome, asthma, ataxia telangiectasia, autoimmune polyglandular syndrome, Burkitt lymphoma, DiGeorge syndrome, chronic myeloid leukemia, or HIV/AIDS) in a subject, by increasing the immunity of the subject.

In some cases, the methods provided herein can be used to reduce the likelihood of progression of a disease or clinical condition (e.g., an immune related disorder such as, without limitation, Addison disease, Celiac disease, Dermatomyositis, Graves’ disease, Hashimoto thyroiditis, multiple sclerosis, myasthenia gravis, pernicious anemia, reactive arthritis, rheumatoid arthritis, Sjogren syndrome, type 1 diabetes, psoriasis, irritable bowel syndrome, asthma, ataxia telangiectasia, autoimmune polyglandular syndrome, Burkitt lymphoma, DiGeorge syndrome, chronic myeloid leukemia, or HIV/AIDS) in a subject, by increasing the immunity of the subject.

The methods can include administering to a subject an effective amount of a composition provided herein, where the composition includes beta glucan, quercetin, and one or more of vitamin A, vitamin C, vitamin D, and zinc (e.g., where the beta glucan is in the form of β-(1,3)-glucan and the quercetin has a bioavailability of greater than 1). An effective amount of a composition can be, for example, an amount that is effective to increase immune function in a subject to which the composition is administered. For example, immune function in a subject to which the composition is administered can be increased by at least 5% (e.g., at least 10%, at least 20%, at least 25%, at least 30%, at least 50%, or at least 75%). Immune function can be assessed using any appropriate method. For example, immune function can be assessed by measuring B cell activity and/or B cell count, T cell activity and/or T cell count, T-cell dependent antibody response, natural killer cell cytotoxicity, and/or a cytokine response). Effectiveness also can be assessed based on the incidence rate of an immune disorder in subjects administered a composition provided herein versus the incidence rate of the immune disorder in subjects administered a control composition that lacks beta glucan and/or quercetin as included in the presently described compositions In some cases, an effective amount can be a daily dose as set forth in the tables herein.

### Exemplary Embodiments

Embodiment 1 is a composition comprising (a) beta glucan, (b) quercetin, and (c) one or more of: vitamin A, vitamin C, vitamin D, and zinc.

Embodiment 2 is the composition of embodiment 1, wherein the beta glucan is in the form of β-(1,3)-glucan.

Embodiment 3 is the composition of embodiment 1 or 2, wherein the quercetin is in a blend comprising a Sophora japonica extract and one or more phosopholipids.

Embodiment 4 is the composition of any one of embodiments 1-3, wherein the quercetin is complexed to one or more phospholipids.

Embodiment 5 is the composition of any one of embodiments 1-4, wherein the composition comprises vitamin A.

Embodiment 6 is the composition of any one of embodiments 1-5, wherein the composition comprises vitamin C.

Embodiment 7 is the composition of any one of embodiments 1-6, wherein the composition comprises vitamin D.

Embodiment 8 is the composition of any one of embodiments 1-7, wherein the composition comprises zinc.

Embodiment 9 is the composition of any one of embodiments 1-8, wherein the composition comprises vitamin A, vitamin C, vitamin D, and zinc.

Embodiment 10 is the composition of embodiment 8, wherein the zinc is in the form of an amino acid chelated zinc.

Embodiment 11 is a composition comprising beta glucan, quercetin, vitamin D, vitamin A, vitamin C, and zinc.

Embodiment 12 is the composition of embodiment 11, wherein the beta glucan is in the form of β-(1,3)-glucan.

Embodiment 13 is the composition of embodiment 11 or embodiment 12, wherein the quercetin is in a blend comprising a Sophora japonica extract and one or more phosopholipids.

Embodiment 14 is the composition of any one of embodiments 11-13, wherein the quercetin is complexed to one or more phospholipids.

Embodiment 15 is the composition of any one of embodiments 11-14, wherein the zinc is a zinc-amino acid compound.

Embodiment 16 is the composition of any one of embodiments 11-15, wherein the zinc is a zinc-amino acid compound conjugated with a polysaccharide.

Embodiment 17 is the composition of any one of embodiments 11-16, wherein the beta glucan is present in an amount of about 35 mg to about 1500 mg.

Embodiment 18 is the composition of any one of embodiments 11-17, wherein the quercetin is present in an amount of about 5 mg to about 1500 mg.

Embodiment 19 is the composition of any one of embodiments 11-18, wherein the vitamin A is present in an amount of about 25 mcg RAE to about 1500 mcg RAE.

Embodiment 20 is the composition of any one of embodiments 11-19, wherein the vitamin C is present in an amount of about 9 mg to about 2000 mg.

Embodiment 21 is the composition of any one of embodiments 11-20, wherein the vitamin D is present in an amount of about 0.005 mg to about 0.1 mg.

Embodiment 22 is the composition of any one of embodiments 11-21, wherein the zinc is present in an amount of about 1 mg to about 75 mg.

Embodiment 23 is the composition of any one of embodiments 11-22, wherein the quercetin has a bioavailability of greater than 1, or about 2 to about 100, or about 25 to about 75, or about 40 to about 60.

Embodiment 24 is the composition of any one of embodiments 11-23, wherein the beta glucan was isolated from algae.

Embodiment 25 is the composition of embodiment 24, wherein the algae is Euglena gracillis algae.

Embodiment 26 is the composition of any one of embodiments 11-25, wherein the composition is in the form of a tablet or capsule.

Embodiment 27 sis a composition comprising (a) beta glucan in an amount of about 35 mg to about 1500 mg, (b) quercetin in an amount of about 5 mg to about 1500 mg, (c) vitamin A in an amount of about 25 mcg RAE to about 1500 mcg RAE mg, (d) vitamin C in an amount of about 9 mg to about 2000 mg, (e) vitamin D in an amount of about 0.005 mg to about 0.1 mg, and (f) zinc in an amount of about 1 mg to about 75 mg.

Embodiment 28 is the composition of embodiment 27, wherein the beta glucan is present in an amount of about 100 mg to about 300 mg.

Embodiment 29 is the composition of embodiment 27 or 28, wherein the quercetin is present in an amount of about 5 mg to about 200 mg.

Embodiment 30 is the composition of any one of embodiments 27-29, wherein the vitamin A is present in an amount of about 400 mcg RAE to about 500 mcg RAE.

Embodiment 31 is the composition of any one of embodiments 27-30, wherein the vitamin C is present in an amount of about 10 mg to about 100 mg.

Embodiment 32 is the composition of any one of embodiments 27-31, wherein the vitamin D is present in an amount of about 0.009 mg to about 0.05 mg.

Embodiment 33 is the composition of any one of embodiments 27-32, wherein the zinc is present in an amount of about 5 mg to about 50 mg.

Embodiment 34 is the composition of any one of embodiments 27-33, wherein the zinc is in the form of an amino acid chelated zinc.

Embodiment 35 is the composition of any one of embodiments 27-34, wherein the quercetin is in a blend comprising a Sophora japonica extract and one or more phosopholipids.

Embodiment 36 is the composition of any one of embodiments 27-35, wherein the quercetin is complexed to one or more phospholipids.

Embodiment 37 is the composition of any one of embodiments 27-36, wherein the beta glucan is in the form of β (1,3)-glucan.

Embodiment 38 is a composition comprising (a) at least about 35 mg of beta glucan, (b) at least about 5 mg of quercetin, (c) at least about 25 mcg RAE of vitamin A, (d) at least about 9 mg of vitamin C, (e) at least about 0.005 mg of vitamin D, and (f) at least about 1 mg of zinc.

The compositions and methods herein will be further described in the following examples, which do not limit the scope of the disclosure.

### EXAMPLES

### Example 1 - An Exemplary Dietary Supplement Composition

A dietary supplement was prepared with the composition shown in Table 1 below. The dietary supplement was prepared as a vegetarian capsule using hydroxypropyl methylcellulose, and further including microcrystalline cellulose and magnesium stearate as binding agents, as well as silicon dioxide as a glidant.

TABLE 1

Exemplary dietary supplement composition.

| | | |
|---|---|---|
| Vitamin A (as retinyl palmitate) | 400-500 | mcg RAE |
| Vitamin C (as ascorbic acid) | 24-30 | mg |
| Vitamin D (as cholecalciferol) | 0.01-0.03 | mg |
| Zinc (as zinc-amino acid oligofructose complex) | 10-12 | mg |
| Beta-Glucan (as β-(1,3)-glucan) | 175-225 | mg |
| Quercetin Blend (includes Sophora japonica extract and phospholipids) | 35-65 | mg |

### Example 2 - An Exemplary Dietary Supplement Composition

A dietary supplement is prepared with the composition shown in Table 2 below.

TABLE 2

Exemplary dietary supplement composition.

| | | |
|---|---|---|
| Vitamin A (as retinyl palmitate) | 400-500 | mcg RAE |
| Vitamin C (as ascorbic acid) | 24-30 | mg |
| Vitamin D (as cholecalciferol) | 0.01-0.03 | mg |
| Zinc (as zinc-amino acid oligofructose complex) | 10-12 | mg |
| Beta-Glucan (as β-(1,3)-glucan) | 30-500 | mg |
| Quercetin Blend (includes Sophora japonica extract and phospholipids) | 500-2000 | mg |

### Example 3 - An Exemplary Dietary Supplement Composition

A dietary supplement is prepared with the composition shown in Table 2 below.

TABLE 3

Exemplary dietary supplement composition.

| | | |
|---|---|---|
| Vitamin A (as retinyl palmitate) | 400-500 | mcg RAE |
| Vitamin C (as ascorbic acid) | 24-30 | mg |
| Vitamin D (as cholecalciferol) | 0.01-0.03 | mg |
| Zinc (as zinc-amino acid oligofructose complex) | 12-16 | mg |
| Beta-Glucan (as β-(1,3)-glucan) | 30-500 | mg |
| Quercetin Blend (includes Sophora japonica extract and phospholipids) | 35-65 | mg |

### Example 4 - An Exemplary Dietary Supplement Composition

A dietary supplement is prepared with the composition shown in Table 2 below.

TABLE 4

Exemplary dietary supplement composition.

| | | |
|---|---|---|
| Vitamin A (as retinyl palmitate) | 400-500 | mcg RAE |
| Vitamin C (as ascorbic acid) | 50-200 | mg |
| Vitamin D (as cholecalciferol) | 0.01-0.03 | mg |
| Zinc (as zinc-amino acid oligofructose complex) | 10-12 | mg |
| Beta-Glucan (as β-(1,3)-glucan) | 30-500 | mg |
| Quercetin Blend (includes Sophora japonica extract and phospholipids) | 35-65 | mg |

### Example 5 - An Exemplary Dietary Supplement Composition

A dietary supplement is prepared with the composition shown in Table 2 below.

TABLE 5

Exemplary dietary supplement composition.

| | | |
|---|---|---|
| Vitamin A (as retinyl palmitate) | 400-500 | mcg RAE |
| Vitamin C (as ascorbic acid) | 24-30 | mg |
| Vitamin D (as cholecalciferol) | 0.01-0.03 | mg |
| Zinc (as zinc-amino acid oligofructose complex) | 12-16 | mg |
| Beta-Glucan (as β-(1,3)-glucan) | 30-500 | mg |
| Quercetin Blend (includes Sophora japonica extract and phospholipids) | 500-2000 | mg |

### Example 6 - An Exemplary Dietary Supplement Composition

A dietary supplement is prepared with the composition shown in Table 2 below.

TABLE 6

Exemplary dietary supplement composition.

| | | |
|---|---|---|
| Vitamin A (as retinyl palmitate) | 400-500 | mcg RAE |
| Vitamin C (as ascorbic acid) | 50-200 | mg |
| Vitamin D (as cholecalciferol) | 0.01-0.03 | mg |
| Zinc (as zinc-amino acid oligofructose complex) | 10-12 | mg |
| Beta-Glucan (as β-(1,3)-glucan) | 30-500 | mg |
| Quercetin Blend (includes Sophora japonica extract and phospholipids) | 500-2000 | mg |

### Example 7 - An Exemplary Dietary Supplement Composition

A dietary supplement is prepared with the composition shown in Table 2 below.

TABLE 7

Exemplary dietary supplement composition.

| | | |
|---|---|---|
| Vitamin A (as retinyl palmitate) | 400-500 | mcg RAE |
| Vitamin C (as ascorbic acid) | 50-200 | mg |
| Vitamin D (as cholecalciferol) | 0.01-0.03 | mg |
| Zinc (as zinc-amino acid oligofructose complex) | 12-16 | mg |
| Beta-Glucan (as β-(1,3)-glucan) | 30-500 | mg |
| Quercetin Blend (includes Sophora japonica extract and phospholipids) | 35-65 | mg |

### Example 8 - An Exemplary Dietary Supplement Composition

A dietary supplement is prepared with the composition shown in Table 2 below.

TABLE 8

Exemplary dietary supplement composition.

| | | |
|---|---|---|
| Vitamin A (as retinyl palmitate) | 400-500 | mcg RAE |
| Vitamin C (as ascorbic acid) | 50-200 | mg |
| Vitamin D (as cholecalciferol) | 0.01-0.03 | mg |
| Zinc (as zinc-amino acid oligofructose complex) | 12-16 | mg |
| Beta-Glucan (as β-(1,3)-glucan) | 30-500 | mg |
| Quercetin Blend (includes Sophora japonica extract and phospholipids) | 500-2000 | mg |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising:
beta glucan,
quercetin, and
one or more of: vitamin A, vitamin C, vitamin D, and zinc.

2. The composition of claim 1, wherein the beta glucan is in the form of β-(1,3)-glucan.

3. The composition of claim 1, wherein the quercetin is in a blend comprising a Sophora japonica extract and one or more phosopholipids.

4. The composition of claim 1, wherein the quercetin is complexed to one or more phospholipids.

5. The composition of claim 1, wherein the composition comprises vitamin A, vitamin C, vitamin D, and zinc.

6. The composition of claim 5, wherein the zinc is in the form of an amino acid chelated zinc.

7. The composition of claim 5, wherein the zinc is a zinc-amino acid compound conjugated with a polysaccharide.

8. The composition of claim 5, wherein the beta glucan is present in an amount of about 35 mg to about 1500 mg, the quercetin is present in an amount of about 5 mg to about 1500 mg, the vitamin A is present in an amount of about 25 mcg RAE to about 1500 mcg RAE, the vitamin C is present in an amount of about 9 mg to about 2000 mg, the vitamin D is present in an amount of about 0.005 mg to about 0.1 mg, and the zinc is present in an amount of about 1 mg to about 75 mg.

9. The composition of claim 5, wherein the quercetin has a bioavailability of greater than 1, or about 2 to about 100, or about 25 to about 75, or about 40 to about 60.

10. The composition of claim 5, wherein the beta glucan was isolated from algae.

11. The composition of claim 10, wherein the algae is Euglena gracillis algae.

12. The composition of claim 5, wherein the composition is in the form of a tablet or capsule.

13. A composition comprising:
(a) beta glucan in an amount of about 35 mg to about 1500 mg,
(b) quercetin in an amount of about 5 mg to about 1500 mg,
(c) vitamin A in an amount of about 25 mcg RAE to about 1500 mcg RAE mg,
(d) vitamin C in an amount of about 9 mg to about 2000 mg,
(e) vitamin D in an amount of about 0.005 mg to about 0.1 mg, and
(f) zinc in an amount of about 1 mg to about 75 mg.

14. The composition of claim 13, wherein the beta glucan is present in an amount of about 100 mg to about 300 mg, the quercetin is present in an amount of about 5 mg to about 200 mg, the vitamin A is present in an amount of about 400 mcg RAE to about 500 mcg RAE, the vitamin C is present in an amount of about 10 mg to about 100 mg, the vitamin D is present in an amount of about 0.009 mg to about 0.05 mg, and the zinc is present in an amount of about 5 mg to about 50 mg.

15. The composition of claim 13, wherein the zinc is in the form of an amino acid chelated zinc.

16. The composition of claim 13, wherein the quercetin is in a blend comprising a Sophora japonica extract and one or more phosopholipids.

17. The composition of claim 13, wherein the quercetin is complexed to one or more phospholipids.

18. The composition of claim 13, wherein the beta glucan is in the form of β (1,3)-glucan.

19. A composition comprising:
(a) at least about 35 mg of beta glucan,
(b) at least about 5 mg of quercetin,
(c) at least about 25 mcg RAE of vitamin A,
(d) at least about 9 mg of vitamin C,
(e) at least about 0.005 mg of vitamin D, and
(f) at least about 1 mg of zinc.
